(19) United States
(12) Patent Application Publication
(10) Pub. No.: US 2015/0366705 A1
(43) Pub. Date: Dec. 24, 2015

(54) OPHTHALMIC TREATMENT APPARATUS AND METHOD FOR CONTROLLING SAME

(71) Applicant: LUTRONIC CORPORATION, Goyang-si, Gyeonggi-do (KR)

(72) Inventors: Tae Ho HA, Goyang (KR); Kwang Su HAN, Goyang (KR); Eun Kyoum KIM, Goyang (KR)

(73) Assignee: Lutronic Corporation, Goyang-si, Gyeonggi-do (KR)

(21) Appl. No.: 14/414,697

(22) PCT Filed: Jul. 15, 2013
(86) PCT No.: PCT/KR2013/006327

§ 371 Date: Jan. 13, 2015

(30) Foreign Application Priority Data
Jul. 13, 2012 (KR) 10-2012-0076780
Jul. 13, 2012 (KR) 10-2012-0076781

(51) Int. Cl. A61F 9/008

(52) U.S. Cl. CPC A61F 9/008; A61F 2009/00851; A61F 2009/00887; A61F 2009/00891; A61F 2009/00844; A61F 2009/00885

(57) ABSTRACT

The present invention relates to an ophthalmic treatment apparatus and to a method for controlling same. The ophthalmic treatment apparatus according to the present invention comprises: a beam generating unit for generating a treatment beam; an image unit for generating an image of an eyeball to be treated and displaying the image of the eyeball; an input unit for applying an input signal so as to display a lesion region in the image of the eyeball displayed by the image unit; and a control unit for controlling the operation of the beam generating unit such that the treatment beam can be radiated onto the lesion region in accordance with the lesion region displayed by means of the input signal inputted by the input unit.

### BACKGROUND OF THE INVENTION

1. Field of the Invention

The present invention relates to an ophthalmic treatment apparatus and a method of controlling the same and, more particularly, to an ophthalmic treatment apparatus for treating ocular diseases and a method of controlling the same.

2. Related Art

An ophthalmic treatment apparatus is an apparatus for radiating beams for treatment in order to treat glaucoma, a cataract, or macular degeneration generated in an eyeball. In this case, from among ocular diseases, glaucoma is attributable to an increase in the intraocular pressure of vitreous humour, a cataract is attributable to the whitening phenomenon of the crystalline lens, and macular degeneration is generated in the retina.

This ophthalmic treatment apparatus captures an image of an eyeball in order to identify a region in which an ocular disease has occurred. Furthermore, the ophthalmic treatment apparatus radiates a beam for treatment to the lesion region of the eyeball based on the captured image of the eyeball.

Meanwhile, a conventional ophthalmic treatment apparatus has been disclosed in “Korean Patent Application Publication No. 2001-0022813” entitled “Medical Laser Guidance Apparatus.” “The medical laser guidance apparatus, that is, the aforementioned prior document, has a technical characteristic that includes a laser light source, laser light guidance means for guiding light from the laser light source to the light path of a retina viewing device, laser light location control means for moving the location of the laser light from the laser light source within the light path, and control means for controlling the laser light source and the location control means in order to guide the laser light to a specific point or region of the retina of an eye.

However, the technical characteristic disclosed in the conventional prior document controls the location to which laser light is radiated using the viewing device and the location control means, but has a disadvantage in that effective treatment is impossible because it is difficult to set an accurate lesion region.

### SUMMARY OF THE INVENTION

An object of the present invention is to provide an ophthalmic treatment apparatus for radiating beams for treatment after accurately specifying a treatment region before radiating the beams for treatment to an ocular disease and a method of controlling the same.

In order to accomplish the object, the present invention provides an ophthalmic treatment apparatus including a beam generation unit generating beams for treatment and a beam delivery unit guiding the beams for treatment to an eyeball, including an image unit which generates an image of the eyeball to be treated and displays the image of the eyeball, an input unit which applies an input signal so that a lesion region of the image of the eyeball displayed by the image unit is displayed, and a control unit which controls the operations of the beam generation unit and the beam delivery unit so that the beams for treatment are radiated to the displayed lesion region based on the lesion region input and displayed by the input unit.

Furthermore, there may be provided a method of controlling an ophthalmic treatment apparatus including a beam generation unit generating beams for treatment and a beam delivery unit guiding the beams for treatment to an eyeball, including steps of (a) generating an image of an eyeball to be treated, (b) displaying the image of the eyeball, (c) drawing a lesion region of the displayed image of the eyeball along an outer circumference of the lesion region, and (d) controlling operations of the beam generation unit and the beam delivery unit so that the beams for treatment are radiated within the drawn lesion region.

Furthermore, there may be provided an ophthalmic treatment apparatus, including a beam generation unit which generates beams for treatment and an alignment beam, a beam delivery unit which guides the beams for treatment and the alignment beam to the treatment region of an eyeball, a tissue detection unit which detects a tissue state according to the alignment beam radiated to the treatment region of the eyeball, and a control unit which controls the operation of the beam delivery unit based on a signal detected by the tissue detection unit so that a location to which the beams for treatment are radiated is controlled.

Furthermore, there may be provided a method of controlling an ophthalmic treatment apparatus, including a beam generation unit which generates an alignment beam and beams for treatment, a beam delivery unit which guides the alignment beam and the beams for treatment to a treatment region of an eyeball, and a tissue detection unit which detects whether a tissue state in the treatment region of the eyeball to which the alignment beam has been radiated is changed, including steps of (a) generating the alignment beam and radiating the generated alignment beam to the treatment region of the eyeball, (b) detecting whether the tissue state in the treatment region of the eyeball to which the alignment beam has been radiated is changed, and (c) controlling an operation of the beam delivery control unit 1700 based on whether the tissue state in the treatment region of the eyeball to which the alignment beam has been radiated is changed so that a location to which the beams for treatment are radiated in the treatment region of the eyeball is changed.

The ophthalmic treatment apparatuses and the methods of controlling the same according to the present invention are advantageous in that accurate treatment is possible and treatment efficiency can be improved because an image of the subject to be treated is captured and the location to which a beam for treatment is radiated is determined based the captured image or the radiation location is determined using tissue characteristics.

### BRIEF DESCRIPTION OF THE DRAWINGS

FIG. 1 is a schematic configuration of an ophthalmic treatment apparatus in accordance with a first embodiment of the present invention,

FIG. 2 is a control block diagram of the ophthalmic treatment apparatus in accordance with the first embodiment of the present invention,

FIG. 3 is a schematic configuration in which an image of one region of an eyeball captured by the image unit of the ophthalmic treatment apparatus in accordance with the first embodiment of the present invention is displayed,

FIG. 4 is a schematic configuration in which beams for treatment are radiated within the lesion region of an eyeball,

FIG. 5 is a control flowchart illustrating the ophthalmic treatment apparatus in accordance with the first embodiment of the present invention,

FIG. 6 is a schematic configuration of an ophthalmic treatment apparatus in accordance with second and third embodiments of the present invention,

FIG. 7 is a control block diagram of the ophthalmic treatment apparatus in accordance with the second and the third embodiments of the present invention,

FIG. 8 is a control flowchart illustrating a method of controlling the ophthalmic treatment apparatus in accordance with the second embodiment of the present invention, and

FIG. 9 is a control flowchart illustrating a method of controlling the ophthalmic treatment apparatus in accordance with the third embodiment of the present invention.

### DESCRIPTION OF EXEMPLARY EMBODIMENTS

Hereinafter, ophthalmic treatment apparatuses and methods of controlling the same in accordance with embodiments of the present invention are described in detail with reference to the accompanying drawings.

Prior to a description, the ophthalmic treatment apparatuses and the methods of controlling the same in accordance with the embodiments of the present invention have been illustrated as treating a lesion generated in the retina, but they may be used to treat lesions generated in an eyeball in addition to the retina.

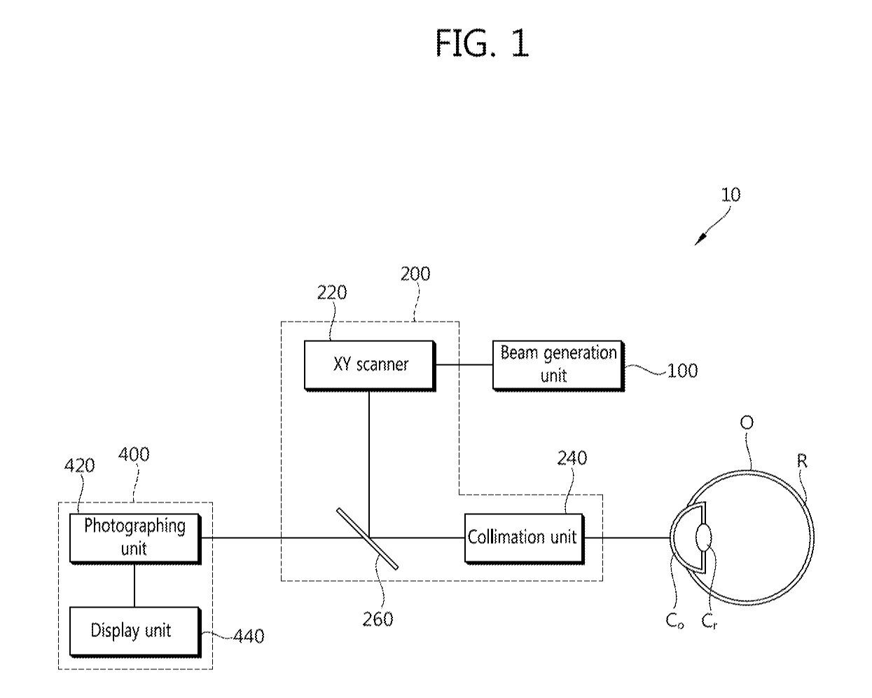

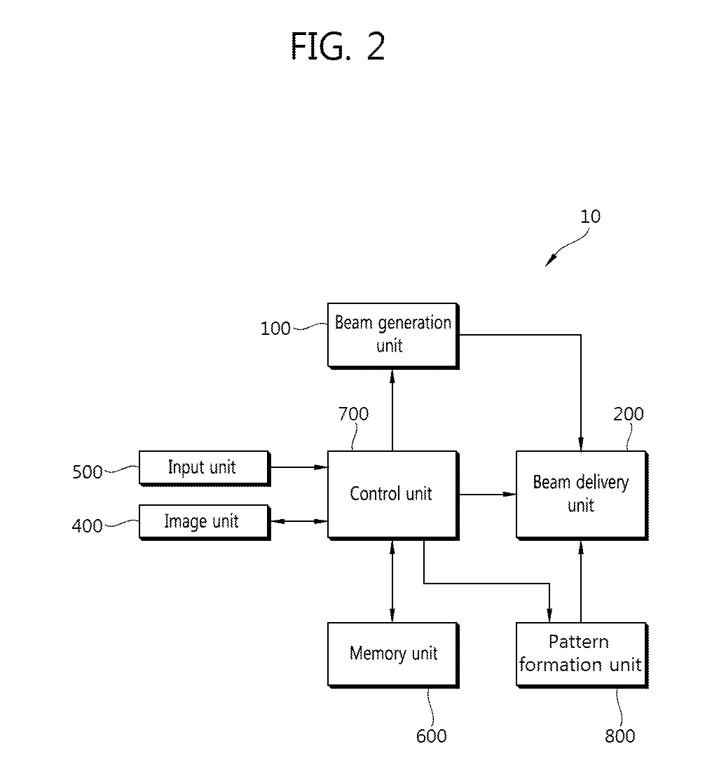

FIG. 1 is a schematic configuration of an ophthalmic treatment apparatus in accordance with a first embodiment of the present invention, and FIG. 2 is a control block diagram of the ophthalmic treatment apparatus in accordance with the first embodiment of the present invention.

As illustrated in FIG. 1 and FIG. 2, the ophthalmic treatment apparatus 10 in accordance with the first embodiment of the present invention includes a beam generation unit 100, a beam delivery unit 200, an image unit 400, an input unit 500, a memory unit 600, and a control unit 700. Furthermore, the ophthalmic treatment apparatus 10 in accordance with an embodiment of the present invention may further include a pattern formation unit 800 that is used when a beam for treatment is radiated to a pattern spot.

The beam generation unit 100 is provided to generate beams for treatment. A laser is used as the beam for treatment generated by the beam generation unit 100. The beam generation unit 100 may be configured to include a laser resonator or laser diode for generating a laser generated as the beam for treatment. The beam generation unit 100 preferably generates a beam for treatment that has a wavelength band according to tissue of an eyeball O.

The beam delivery unit 200 includes an XY scanner 220, a collimation unit 240, and a beam splitter 260. The beam delivery unit 200 guides a beam for treatment, provided by the beam generation unit 100, to the treatment region of the eyeball O.

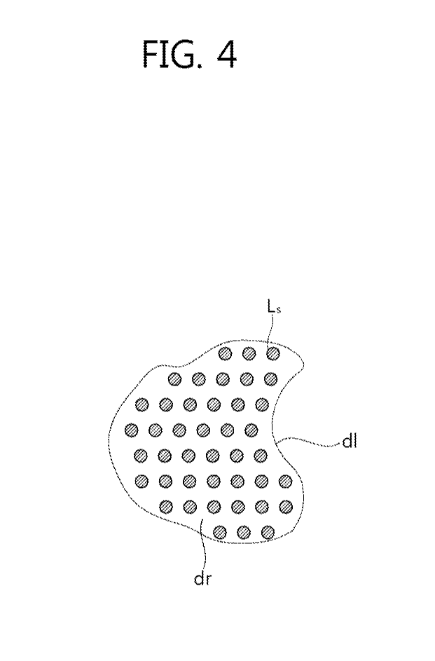

Assuming that an optical axial line in which beams for treatment is radiated to the retina R through a cornea Co and a crystalline lens Cr is a Z axis, the XY scanner 220 is provided to form the spots Ls (refer to FIG. 4) of the beams for treatment on an XY plane, that is, a direction perpendicular to the Z axis. The XY scanner 220 includes a reflection mirror (not illustrated) which rotatably moves along any one of the rotational axial line of an X axis and the rotational axial line of a Y axis and a reflection mirror (not illustrated) which rotatably moves along any one of the rotational axial line of the X axis and the rotational axial line of the Y axis. As described above, the XY scanner 220 includes at least two reflection mirrors and may move the spots Ls of the beams for treatment in the direction of the X axis or Y axis on the XY plane.

The collimation unit 240 is disposed between the eyeball O and the XY scanner, and it collimates a beam for treatment incident from the XY scanner 220 to the treatment region of the eyeball O. The collimation unit 240 is used as an object lens including a plurality of lenses. The beam splitter 260 is disposed between the XY scanner 220 and the collimation unit 240, and it guides a beam for treatment from the XY scanner 220 to the collimation unit 240.

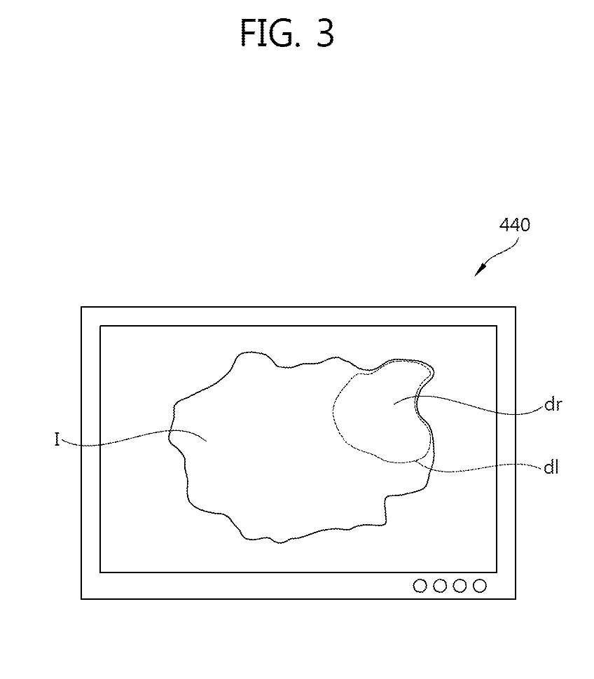

Next, FIG. 3 is a schematic configuration in which an image of one region of an eyeball captured by the image unit of the ophthalmic treatment apparatus in accordance with the first embodiment of the present invention is displayed, and FIG. 4 is a schematic configuration in which beams for treatment are radiated within the lesion region of an eyeball.

As illustrated in FIGS. 3 and 4, the image unit 400 captures the eyeball O, that is, the subject of treatment, and displays the captured image I of the eyeball. The image I of the eyeball that has been captured and formed by the image unit 400 is used to set a lesion region dr. In an embodiment of the present invention, the image unit 400 includes a photographing unit 420 and a display unit 440.

The photographing unit 420 is used to photograph a shape of the eyeball O, more specifically, the lesion region dr, that is, a disease generated in the eyeball O. The photographing unit 420 may include at least one of Optical Coherence Tomography (OCT) and a digital camera for performing a computed tomography scan on the eyeball O. Furthermore, the photographing unit 420 may include a variety of types of known photographing equipment in addition to the aforementioned OCT and digital camera.

The display unit 440 is used to display the image I of the eyeball, captured and formed by the photographing unit 420, to an operator. As illustrated in FIG. 3, the display unit 440 displays the image I of the eyeball that has been captured and formed by the photographing unit 420. The image I of the eyeball that is first displayed on the display unit 440 is a specific region of the eyeball O that includes the lesion region dr.

The input unit 500 applies an input signal so that the lesion region dr that belongs to the image I of the eyeball displayed by the image unit 400 is displayed. That is, the input unit 500 applies an input signal so that the lesion region dr is formed along a drawing line dl, that is, the outer circumference of the lesion region dr that belongs to the image I of the eyeball. In an embodiment of the present invention, the input unit 500 may be any one of a mouse and a digitizer for moving along the outer circumference of the lesion region dr, drawing the lesion region dr, and applying the input signal.

Meanwhile, the display unit 440 may further include a touch panel (not illustrated). The touch panel is disposed in a display region displayed by the display unit 440. The input unit 500 is provided to draw the lesion region dr in response to the touch panel, that is, to set the drawing line dl. For example, if the touch panel is a capacitive input method, a capacitive pen, etc. may be used. If the touch panel is a resistive type input method, a pressure pen, etc. may be used.

In another embodiment, the drawing line dl may be input along the outer circumference of the lesion region dr without using the input unit 500. If the display unit 440 includes a touch panel using a capacitive input method, the drawing line dl of the lesion region may be formed in response to the contact or motion of an operator's hand.

The memory unit 600 stores the shape of the lesion region dr drawn by the input unit 500. Information about the shape of the lesion region dr that has been stored in the memory unit 600 is transmitted to the control unit 700. The control unit 700 radiates beams for treatment within the shape of the lesion region dr based on the information about the shape of the lesion region dr stored in the memory unit 600 as described above.

Next, the control unit 700 controls the operations of the beam generation unit 100 and the beam delivery unit 200 so that the beams for treatment are radiated within the displayed lesion region dr based on the lesion region dr that has been input by the input unit 500 and displayed. The control unit 700 receives information about a shape of the lesion region dr, formed in response to input from the input unit 500, from the memory unit 600 and controls the operations of the beam generation unit 100 and the beam delivery unit 200 so that the beams for treatment are radiated within the lesion region dr.

The beams for treatment radiated through the beam generation unit 100 and the beam delivery unit 200 are radiated within the lesion region dr under the control of the control unit 700. In this case, each of the spots Ls of the respective beams for treatment may be radiated in a specific size, and the spots Ls may be radiated within the lesion region dr at specific intervals.

Alternatively, the pattern formation unit 800 may separately form a pattern in which beams for treatment are radiated. The control unit 700 may perform control so that the beams for treatment are radiated within the lesion region based on the pattern formed by the pattern formation unit 800. In this case, the pattern formation unit 800 may configure the spot sizes of the respective beams for treatment or the arrangement of the spots of the beams in various ways. For example, each of beams for treatment may have a square pattern within a lesion region or may have a radial pattern. Alternatively, the radiation density of beams or the spot size of a beam may be great in a portion placed at the center of a lesion region, and the radiation density of beams or the spot size of a beam may be small in a portion placed at the edge.

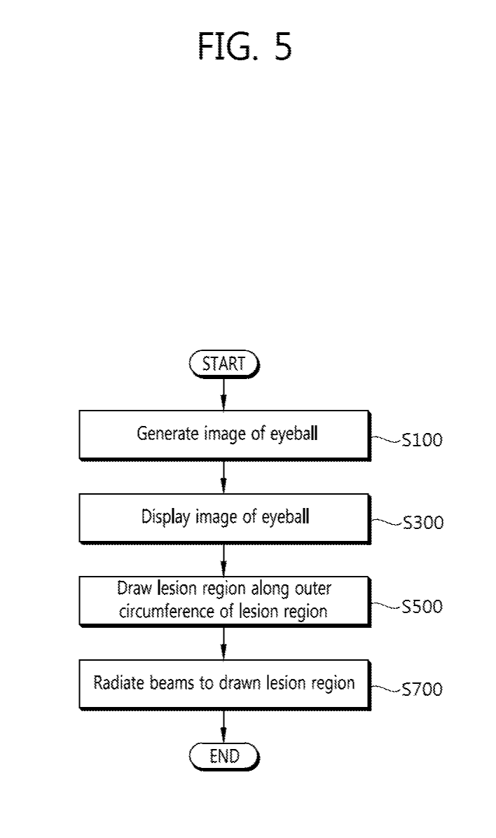

Finally, FIG. 5 is a control flowchart illustrating the ophthalmic treatment apparatus in accordance with the first embodiment of the present invention.

A method of controlling the ophthalmic treatment apparatus 10 configured as described above in accordance with an embodiment of the present invention is described below with reference to FIG. 5.

First, the photographing unit 420 of the image unit 400 operates and forms the image I of the eyeball O by photographing the eyeball O, that is, the subject of treatment (S100). The image I of the eyeball that has been captured and formed by the photographing unit 420 of the image unit 400 is displayed on the display unit 440 of the image unit 400 (S300).

An operator applies an input signal using the input unit 500 so that the drawing line dl is formed along the outer circumference of the lesion region dr that belongs to the image I of the eyeball displayed on the display unit 440 (S500). In this case, the input unit 500 may be any one of a mouse and a digitizer. If the display unit 440 includes a touch panel, a capacitive pen, a resistive pen or the like may be used.

After drawing the lesion region dr on the image I of the eyeball, a shape of the drawn lesion region dr is stored in the memory unit 600. The control unit 700 radiates beams for treatment within the drawn lesion region dr using information about the shape of the lesion region dr that has been stored in the memory unit 600 (S700). In this case, the spots Ls of the beams for treatment may be radiated as respective spots or as a pattern spot. If the spots Ls of the beams for treatment are radiated as a pattern spot, the shape of a pattern spot stored in the pattern formation unit 800 is used.

As described above, an image of an eyeball, that is, the subject of treatment, is formed by photographing the eyeball, a lesion region is set by drawing the lesion region on the image of the eyeball, and beams for treatment are radiated within the set lesion region. Accordingly, an eyeball can be precisely treated, and treatment efficiency can also be improved.

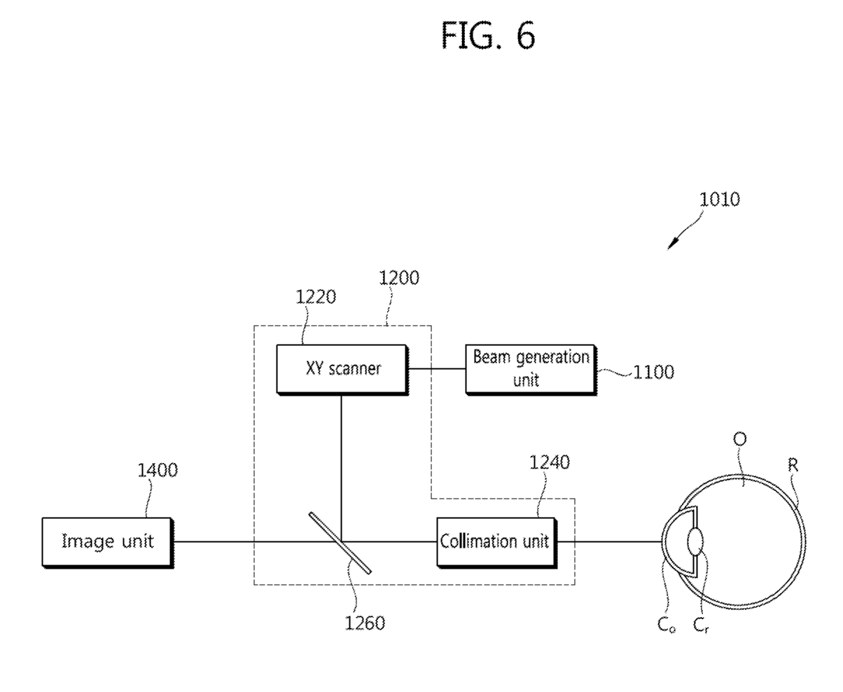

Hereinafter, an ophthalmic treatment apparatus and methods of controlling the same in accordance with second and third embodiments of the present invention are described in detail with reference to FIGS. 6 to 9.

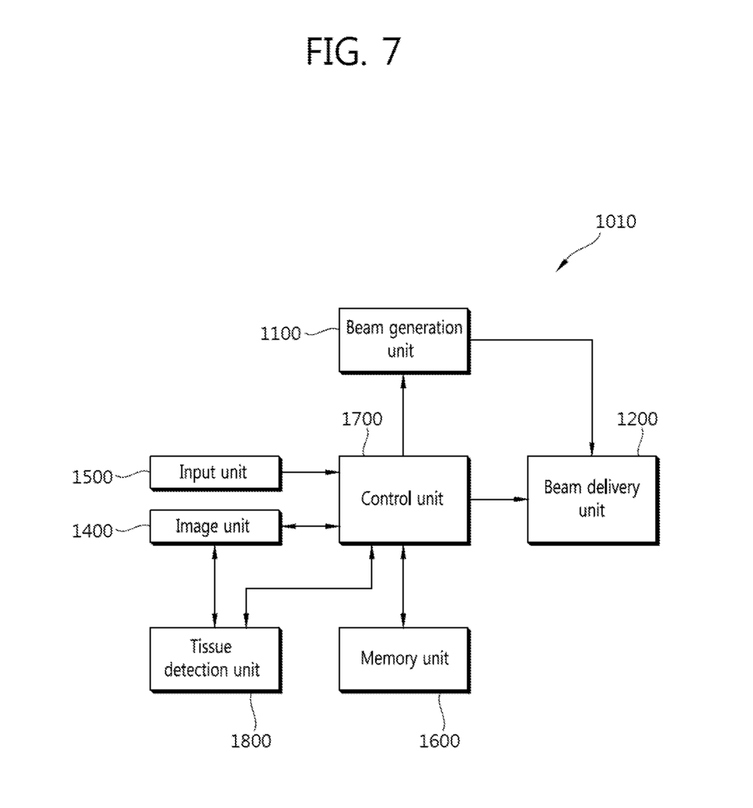

FIG. 6 is a schematic configuration of the ophthalmic treatment apparatus in accordance with the second and the third embodiments of the present invention, and FIG. 7 is a control block diagram of the ophthalmic treatment apparatus in accordance with the second and the third embodiments of the present invention.

As illustrated in FIGS. 6 and 7, the ophthalmic treatment apparatus 1010 in accordance with the second and the third embodiments of the present invention includes a beam generation unit 1100, a beam delivery unit 1200, an image unit 1400, an input unit 1500, a tissue detection unit 1800, a memory unit 1600, and a control unit 1700. The ophthalmic treatment apparatus 1010 in accordance with the embodiments of the present invention may be widely applied to diseases in the cornea Co, the crystalline lens Cr, and the retina R that belong to tissue of an eyeball O.

The beam generation unit 1100 generates beams for treatment and an alignment beam. The beams for treatment and the alignment beam generated by the beam generation unit 1100 oscillate in the form of lasers. In this case, the beams for treatment and the alignment beam generated by the beam generation unit 1100 have different wavelength bands, that is, different types of pulse energy. For example, the wavelength band of the alignment beam generated by the beam generation unit 1100 may have a degree of water sorption higher than that of the beam for treatment.

Since an alignment beam has a wavelength band having a degree of water sorption higher than that of a beam for treatment as described above, a blood vessel region may not be damaged because the alignment beam is absorbed by the blood vessel when the alignment beam is radiated to the blood vessel region of the eyeball O. The beam generation unit 1100 is provided to include an element including a laser diode in order to generate the beams for treatment and the alignment beam.

Meanwhile, the beam generation unit 1100 in the embodiments of the present invention has been illustrated as generating beams for treatment and an alignment beam, but an alignment beam generation unit (not illustrated) may be provided in order to generate an alignment beam independently of beams for treatment.

The beam delivery unit 1200 includes an XY scanner 1220, a collimation unit 1240, and a beam splitter 1260. The beam delivery unit 1200 guides the beams for treatment and the alignment beam to the treatment region of the eyeball O.

Assuming that an optical axial line in which an alignment beam and beams for treatment are radiated to the cornea Co, the crystalline lens Cr, or the retina R is a Z axis, the XY scanner 1220 guides the beams for treatment and the alignment beam to an XY plane, that is, a direction perpendicular to the Z axis. The XY scanner 1220 includes at least 7 reflection mirrors (not illustrated). The at least 7 reflection mirrors of the XY scanner 1220 rotatably move in one of the rotational axial line of an X axis and the rotational axial line of a Y axis, rotatably move in the other of the rotational axial line of the X axis and the rotational axial line of the Y axis, and guide the beams for treatment and the alignment beam, provided by the beam generation unit 1100, in the X axis and the Y axis on the XY plane.

The collimation unit 1240 is provided adjacent to the eyeball O. The collimation unit 1240 collimates beams for treatment and an alignment beam, provided by the XY scanner 1220, to the treatment region of the eyeball O. The collimation unit 1240 is used as an object lens including a plurality of lenses. The beam splitter 1260 is disposed between the XY scanner 1220 and the collimation unit 1240, and it guides the beams for treatment and the alignment beam from the XY scanner 1220 to the collimation unit 1240.

The image unit 1400 is provided to photograph the treatment region of the eyeball O to which beams for treatment and an alignment beam are radiated. The image unit 1400 forms an image of the eyeball O by photographing the treatment region of the eyeball O. The image of the eyeball O formed by the image unit 1400 as described above is viewed by an operator so that treatment efficiency can be improved. Known photographing equipment, such as Optical Coherence Tomography (OCT) and a digital camera may be used as the image unit 1400 that is used in the embodiments of the present invention.

The input unit 1500 applies operation signals so that the beam generation unit 1100 and the beam delivery unit 1200 are driven. The input unit 1500 applies an operation signal so that beams for treatment and an alignment beam can be generated by the beam generation unit 1100. The operation signal applied by the input unit 1500 is transmitted to the control unit 1700. The control unit 1700 generates a control signal for controlling the operation of the beam generation unit 1100. Furthermore, the input unit 1500 applies an input signal so that the beam delivery unit 1200 controls the location to which beams for treatment and an alignment beam are radiated. In this case, the operation signal applied by the input unit 1500 is transmitted to the control unit 1700. The control unit 1700 generates a control signal for controlling the operation of the beam delivery unit 1200.

Next, the tissue detection unit 1800 detects a tissue state according to an alignment beam radiated to the treatment region of the eyeball O. For example, the tissue detection unit 1800 detects the generation of a bubble when an alignment beam is radiated to a specific tissue of the eyeball O or detects the absorption of an alignment beam when the alignment beam is radiated to a specific tissue of the eyeball. An optical sensor, etc. capable of detecting the tissue state of the eyeball O according to the radiation of an alignment beam may be used as the tissue detection unit 1800.

The memory unit 1600 stores a signal detected by the tissue detection unit 1800 and a specific region of the eyeball O in which a signal has been detected by the tissue detection unit 1800. In contrast, the memory unit 1600 stores a specific region of the eyeball to which an alignment beam has been radiated and in which a change of the tissue state is not detected by the tissue detection unit 1800. As described above, information stored in the memory unit 1600 is used in methods of controlling the ophthalmic treatment apparatus 1010 to be described later in accordance with the second and the third embodiments of the present invention.

Finally, the control unit 1700 controls the operation of the beam delivery unit 1200 so that beams for treatment are radiated based on a detection signal and corresponding region stored in the memory unit 1600. More specifically, the control unit 1700 receives information which includes the detection of a change of tissue attributable to an alignment beam and a corresponding region of the eyeball O and controls the operation of the beam delivery unit 1200 so that beams for treatment are radiated to the corresponding region of the eyeball O.

Meanwhile, the control unit 1700 controls the operation of the beam delivery unit 1200 based on information stored in the memory unit 1600 so that beams for treatment are not radiated to a corresponding specific region of the eyeball O. More specifically, if information stored in the memory unit 1600 includes no change of tissue attributable to the radiation of an alignment beam, for example, includes a region in which an alignment beam is absorbed, such as a blood vessel region, the control unit 1700 determines that the corresponding region is a region to which beams for treatment are not radiated. The control unit 1700 controls the operation of the beam delivery unit 1200 based on this information stored in the memory unit 1600 so that beams for treatment avoid the corresponding region of the eyeball O without being radiated to the corresponding region.

### Second Embodiment

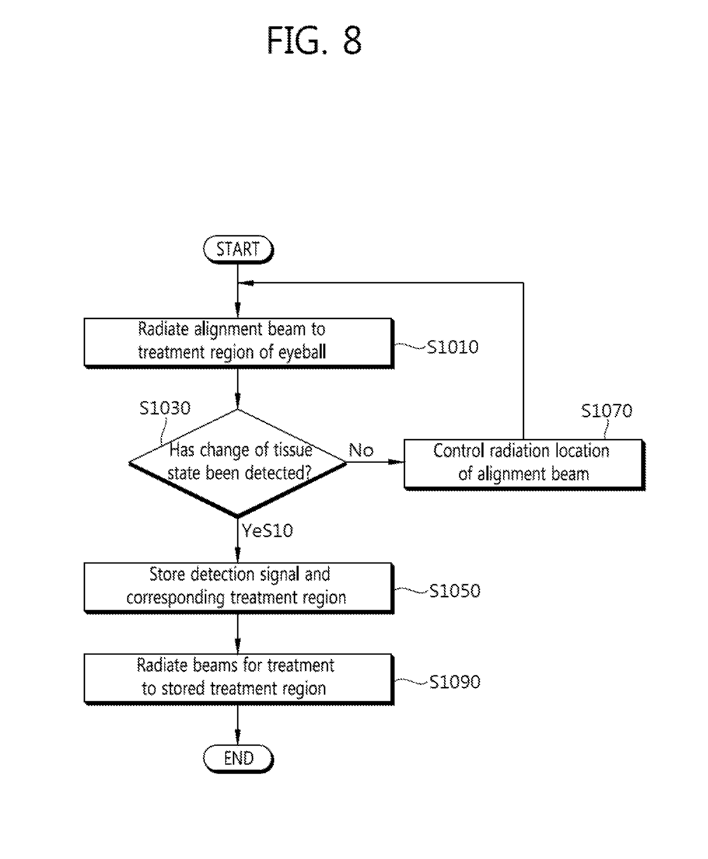

FIG. 8 is a control flowchart illustrating a method of controlling the ophthalmic treatment apparatus in accordance with the second embodiment of the present invention.

The method of controlling the ophthalmic treatment apparatus 1010 in accordance with the second embodiment of the present invention is described below with reference to FIG. 8.

First, the image unit 1400 operates and forms an image of the eyeball O by photographing the eyeball O. The beam generation unit 1100 generates an alignment beam and radiates the alignment beam to the treatment region of the eyeball O (S1010). In this case, the alignment beam preferably has a wavelength band different from that of beams for treatment and has a wavelength band having a high degree of water sorption.

When the alignment beam is radiated to the treatment region of the eyeball O at step ‘S1010’, whether the tissue state of the eyeball O is changed is detected (S1030). If a change of the tissue state in the treatment region of the eyeball O to which the alignment beam has been radiated by the tissue detection unit 1800 is detected at step ‘S1030,’ a corresponding treatment region of the eyeball O that corresponds to a signal detected by the tissue detection unit 1800 is stored in the memory unit 1600 (S1050).

Beams for treatment are radiated using information stored in the memory unit 1600 at step ‘S1050.’ In this case, the beams for treatment are radiated to the corresponding treatment region of the eyeball O, corresponding to the information stored in the memory unit 1600, under the control of the beam delivery unit 1200 (S1090). Meanwhile, if a change of the tissue state in the treatment region of the eyeball O to which the alignment beam has been radiated is not detected at step ‘S1030,’ the location to which the beams for treatment are radiated is not stored, and the radiation location of the alignment beam in the treatment region of the eyeball O is controlled (S1070).

### Third Embodiment

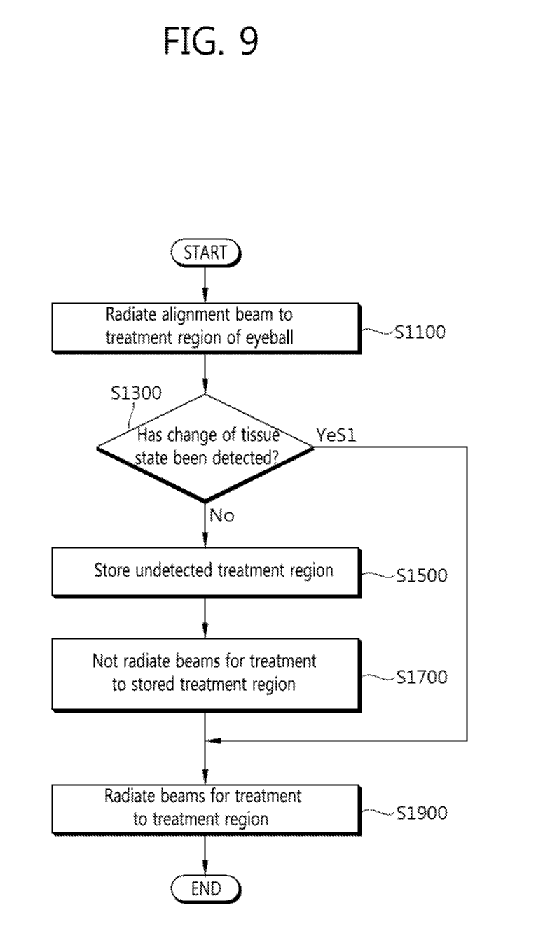

FIG. 9 is a control flowchart illustrating a method of controlling the ophthalmic treatment apparatus in accordance with the third embodiment of the present invention.

The method of controlling the ophthalmic treatment apparatus 1010 in accordance with the third embodiment of the present invention is described below in detail with reference to FIG. 9.

First, when the image unit 1400 is driven, it generates an image of the eyeball O by photographing the eyeball O. Furthermore, an alignment beam is radiated to the treatment region of the eyeball O based on the image of the eyeball O generated by the image unit 1400 (S1100). In this case, as in the second embodiment, the alignment beam radiated to the treatment region of the eyeball O preferably has a wavelength band different from that of beams for treatment and has a wavelength band having a high degree of water sorption.

When the alignment beam is radiated, the tissue detection unit 1800 detects whether the tissue state is changed in the treatment region of the eyeball O to which the alignment beam has been radiated (S1300). If no change of the tissue state is detected at step ‘S1300,’ the treatment region of the eyeball O not detected by the tissue detection unit 1800 is stored in the memory unit 1600 (S1500). The control unit 1700 avoids the radiation of beams for treatment to the corresponding treatment region of the eyeball, not detected by the tissue detection unit 1800, based on information stored in the memory unit 1600 (S1700).

Furthermore, the control unit 1700 radiates the beams for treatment to the treatment region of the eyeball O other than the corresponding treatment region of the eyeball O to which the beams for treatment are not radiated (S1900). In contrast, if a change of the tissue state is detected in the treatment region of the eyeball O to which the alignment beam has been radiated at step ‘S1300,’ the process proceeds to step ‘S1900’ in which the beams for treatment are radiated to the treatment region of the eyeball O in which a change of the tissue state has been detected.

As described above, the treatment region of an eyeball to which beams for treatment are radiated can be measured because whether the tissue state of the eyeball is changed is detected using an alignment beam. Accordingly, treatment efficiency of an eyeball can be improved by accurately radiating beams for treatment.

As described above, although the embodiments of the present invention have been described with reference to the accompanying drawings, those skilled in the art to which the present invention pertains will understand that the present invention may be implemented in other detailed forms without changing the technical spirit or essential characteristics of the present invention.

For example, the aforementioned embodiments have been divided into the first embodiment, the second embodiment, and the third embodiment and described, but the location to which beams for treatment are radiated may be set by combining the first to the third embodiments. More specifically, as in the first embodiment, a lesion region may be set based on an image of the ocular fundus of an eyeball, a pattern in which beams for treatment are radiated may be set, and a tissue characteristic of the corresponding lesion region may be detected by radiating an alignment beam. As in the second embodiment, a region to which the beams for treatment are to be radiated may be stored. As in the third embodiment, regions to which the beams for treatment are to be not radiated may be stored. Furthermore, the control unit may radiate the beams for treatment to the corresponding lesion region based on the pattern set by the pattern formation unit. In this case, the control unit may be controlled so that the beams for treatment are radiated to only the region to which the beams for treatment are to be radiated, which has been obtained using the method of the second embodiment, and the beams for treatment are precluded from being radiated to the regions to which the beams for treatment are to be not radiated, which have been obtained using the method of the third embodiment.

As described above, it is to be understood that the aforementioned embodiments are illustrative and not limitative from all aspects. The scope of the present invention is defined by the appended claims rather than the detailed description, and the present invention should be construed as covering all modifications or variations derived from the meaning and scope of the appended claims and their equivalents.

What is claimed is:

1. An ophthalmic treatment apparatus comprising a beam generation unit generating beams for treatment and a beam delivery unit guiding the beams for treatment to an eyeball, the ophthalmic treatment apparatus comprising:
an image unit which generates an image of the eyeball to be treated and displays the image of the eyeball;
an input unit which applies an input signal so that a lesion region of the image of the eyeball displayed by the image unit is displayed; and
a control unit which controls operations of the beam generation unit and the beam delivery unit so that the beams for treatment are radiated to the displayed lesion region based on the lesion region input and displayed by the input unit.

2. The ophthalmic treatment apparatus of claim 1, wherein the input unit applies the input signal so that a region to which the beams for treatment are radiated is displayed along an outer circumference of the lesion region.

3. The ophthalmic treatment apparatus of claim 2, wherein the input unit comprises at least any one of a mouse and a digitizer which move along the outer circumference of the lesion region and display the region to which the beams for treatment are radiated.

4. The ophthalmic treatment apparatus of claim 1, wherein the image unit comprises:
a photographing unit which generates the image of the eyeball by photographing the eyeball; and
a display unit which displays the image of the eyeball generated by the photographing unit.

5. The ophthalmic treatment apparatus of claim 4, wherein:
the display unit comprises a touch panel, and
the input unit comes in contact with the touch panel, moves along an outer circumference of the lesion region, and forms a region to which the beams for treatment are radiated.

6. The ophthalmic treatment apparatus of claim 5, wherein:
the touch panel adopts a capacitive input method, and
the region to which the beams for treatment are radiated in the lesion region is formed in response to a contact or motion of an operator's hand.

7. A method of controlling an ophthalmic treatment apparatus comprising a beam generation unit generating beams for treatment and a beam delivery unit guiding the beams for treatment to an eyeball, the method comprising steps of:
(a) generating an image of an eyeball to be treated;
(b) displaying the image of the eyeball;
(c) drawing a lesion region of the displayed image of the eyeball along an outer circumference of the lesion region; and
(d) controlling operations of the beam generation unit and the beam delivery unit so that the beams for treatment are radiated within the drawn lesion region.

8. The method of claim 7, wherein the ophthalmic treatment apparatus further comprises:
a photographing unit which generates the image of the eyeball by photographing the eyeball at the step (a); and
a display unit which displays the image of the eyeball generated by the photographing unit at the step (b).

9. The method of claim 7, wherein the step (c) is performed in response to input from at least any one of a mouse and a digitizer.

10. The method of claim 8, wherein:
the display unit comprises a touch panel, and
the step (c) is performed in response to input according to a contact on the touch panel.

11. An ophthalmic treatment apparatus, comprising:
a beam generation unit which generates beams for treatment and an alignment beam;
a beam delivery unit which guides the beams for treatment and the alignment beam to a treatment region of an eyeball;
a tissue detection unit which detects a tissue state according to the alignment beam radiated to the treatment region of the eyeball; and
a control unit which controls an operation of the beam delivery unit based on a signal detected by the tissue detection unit so that a location to which the beams for treatment are radiated is controlled.

12. The ophthalmic treatment apparatus of claim 11, further comprising a memory unit which stores the signal detected by the tissue detection unit and the treatment region in which the signal is detected by the tissue detection unit.

13. The ophthalmic treatment apparatus of claim 12, wherein the control unit controls the operation of the beam delivery unit based on the detection signal and corresponding treatment region stored in the memory unit so that the beams for treatment are radiated.

14. The ophthalmic treatment apparatus of claim 11, further comprising a memory unit which stores a treatment region to which the alignment beam has been radiated and in which a change of a tissue state is not detected by the tissue detection unit.

15. The ophthalmic treatment apparatus of claim 14, wherein the control unit controls the operation of the beam delivery unit based on information stored in the memory unit so that the beams for treatment are not radiated to the corresponding treatment region.

16. The ophthalmic treatment apparatus of claim 11, wherein the alignment beam has a wavelength band having a degree of water sorption higher than a wavelength band of the beam for treatment.

17. A method of controlling an ophthalmic treatment apparatus, comprising a beam generation unit which generates an alignment beam and beams for treatment, a beam delivery unit which guides the alignment beam and the beams for treatment to a treatment region of an eyeball, and a tissue detection unit which detects whether a tissue state in the treatment region of the eyeball to which the alignment beam has been radiated is changed, the method comprising steps of:
(a) generating the alignment beam and radiating the generated alignment beam to the treatment region of the eyeball;
(b) detecting whether the tissue state in the treatment region of the eyeball to which the alignment beam has been radiated is changed; and
(c) controlling an operation of the beam delivery control unit 1700 based on whether the tissue state in the treatment region of the eyeball to which the alignment beam has been radiated is changed so that a location to which the beams for treatment are radiated in the treatment region of the eyeball is changed.

18. The method of claim 17, wherein:
the ophthalmic treatment apparatus further comprises a memory unit, and
the method further comprises a step of storing the signal detected by the tissue detection unit and the treatment region in which the signal has been detected in the memory unit between the step (b) and the step (c).

19. The method of claim 18, wherein the step (c) comprises a step of controlling the operation of the beam delivery control unit based on information about the treatment region stored in the memory unit so that the beams for treatment are radiated to the corresponding treatment region.

20. The method of claim 17, wherein:
the ophthalmic treatment apparatus further comprises a memory unit, and
the method further comprises a step of storing a location of a treatment region that has not been detected by the tissue detection unit if a change of the tissue state in the treatment region is not detected when the alignment beam is radiated between the step (b) and the step (c).

21. The method of claim 20, wherein the step (c) comprises a step of controlling the operation of the beam delivery control unit based on information about the treatment region stored in the memory unit so that the beams for treatment are not radiated to the corresponding treatment region.
